(19) United States
(12) Patent Application Publication
(10) Pub. No.: US 2022/0068506 A1
(43) Pub. Date: Mar. 3, 2022

(54) TELE-COLLABORATION DURING ROBOTIC SURGICAL PROCEDURES

(71) Applicant: Asensus Surgical US, Inc., DURHAM, NC (US)

(72) Inventor: Michael Bruce Wiggin, Raleigh, NC (US)

(21) Appl. No.: 17/460,128

(22) Filed: Aug. 27, 2021

Related U.S. Application Data
(60) Provisional application No. 63/071,332, filed on Aug. 27, 2020.

(51) Int. Cl. G16H 80/00; G16H 40/20; G16H 40/67; G16H 20/40; A61B 90/00; A61B 34/10; A61B 34/30; A61B 34/00

(52) U.S. Cl. CPC G16H 80/00; G16H 40/20; G16H 40/67; G16H 20/40; A61B 90/37; A61B 2034/301; A61B 34/30; A61B 34/70; A61B 34/25; A61B 2034/101; A61B 2090/365; A61B 34/10

(57) ABSTRACT

A telecollaboration system for use during surgery allows initiation of a video conference session between participants, the participants comprising an on-site user in a surgical operating room, and a remote user not in the surgical operating room. Real time images of patient anatomy during a surgical procedure are captured using an endoscope positioned a patient's body cavity and displayed in real-time to the video conference participants. Real time images of the on-site user and the surgical environment external to the patient are also displayed. Input from remote and/or on site participants is used to generate annotations for display to all participants as overlays on the endoscope images and/or the images of the surgical environment. A user interface allows remote and/or on-site participants to re-orient the external cameras to change the view of the surgeon and/or surgical environment displayed to the participants.

This application claims the benefit of U.S. Provisional Application No. 63/071,332, filed Aug. 27, 2020.

### BACKGROUND

Surgical robotic systems are typically comprised of one or more robotic manipulators and a user interface. The robotic manipulators carry surgical instruments or devices used for the surgical procedure. A typical user interface includes input devices, or handles, manually moveable by the surgeon to control movement of the surgical instruments carried by the robotic manipulators. The surgeon uses the interface to provide inputs into the system and the system processes that information to develop output commands for the robotic manipulator.

In the system illustrated in FIG. 1, a surgeon console 12 has two input devices or handles 17, 18. The input devices are configured to be manipulated by a user to generate signals that are used to command motion of a robotically controlled device in multiple degrees of freedom. In use, the user selectively assigns the two input devices to two of the robotic manipulators 13, 14, 15, allowing surgeon control of two of the surgical instruments 10a, 10b, and 10c disposed at the working site at any given time. To control a third one of the instruments disposed at the working site, one of the two input devices is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the new haptic interface devices, the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices are configured to be manipulated by a user to generate signals that are processed by the system to generate instructions used to command motion of the manipulators in order to move the instruments in multiple degrees of freedom.

The surgical system allows the operating room staff to remove and replace surgical instruments carried by the robotic manipulator, based on the surgical need. Once instruments have been installed on the manipulators, the surgeon moves the input devices to provide inputs into the system, and the system processes that information to develop output commands for the robotic manipulator in order to move the instruments and, as appropriate, operate the instrument end effectors.

At times it may be useful for a surgeon to obtain assistance or input from medical personnel located outside the operating room. This application describes a telecollaboration platform that allows personnel located outside the operating room to observe surgical procedures and to provide feedback to the surgeons performing the procedures.

### BRIEF DESCRIPTION OF THE DRAWINGS

FIG. 1 illustrates a surgical robotic system using a tele-collaboration system.

FIG. 2 is a screen shot of an image display during a tele-collaboration system using the disclosed image, and shows and endoscopic image, an image of a surgeon at a surgeon console, an image of a robotic surgical system positioned for surgery on a patient, and images of two remote participants.

### DETAILED DESCRIPTION

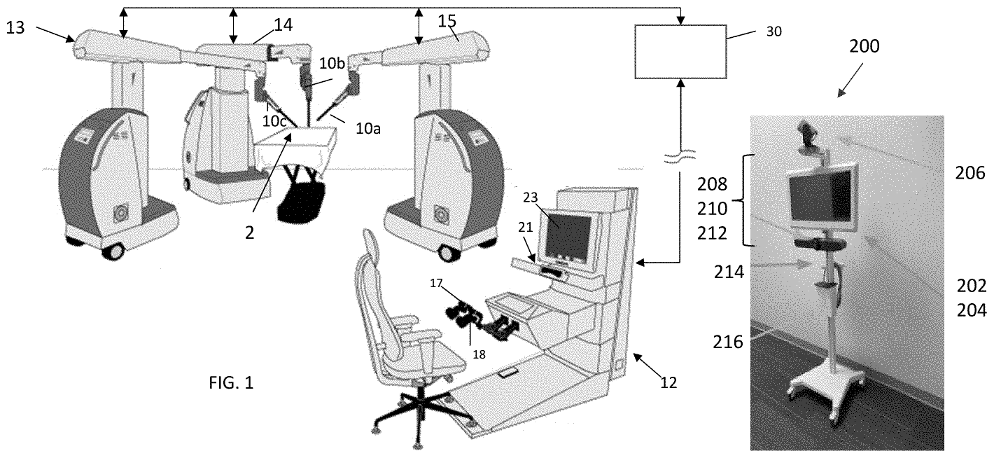

Tele-collaboration system 200 is a system allowing a remotely located user to observe a surgical procedure and to provide real-time input or feedback to personnel performing the procedure. While the system will be described as used with a robot-assisted surgical system such as the one described in connection with FIG. 1, it should be understood that it may be used with other robot-assisted surgical systems, or in other surgical contexts in which robot-assisted systems are not used, such as manual procedures.

Tele-collaboration system 200 allows for real-time collaboration, mentoring, training, proctoring, and observation of surgical procedures from remote locations. This system can simultaneously stream multiple video and endoscope views from the operating room simultaneously, allowing the remote user to view the endoscopic view of relevant portion of the patient anatomy undergoing surgery, and/or areas of the operating room, including, for example, the on-site surgeon, views of the operating room, the robotic surgical system, and the surgeon console.

The system 200 includes a processor 202 and a visual display 204. In the illustrated embodiment, these take the form of a touch screen PC as shown. One or more cameras is positionable within the operating room. These may include a first camera 206 that can be placed to capture images of the operating room, including the manipulator arms of the robotic surgical system, and/or bedside surgical personnel. A second camera 208 may be placed facing the on-site surgeon. Either or both cameras 206, 208 may include pan, tilt and/or zoom capabilities remotely controllable by the remote user as will be described below. Additional cameras may be positioned elsewhere in the operating room. For example, there may be additional cameras at any one or more of the following positions: on one or more of the manipulator arms, on a ceiling mount, or on other fixtures, carts etc. within the operating room. From these positions, cameras can capture images of one or more of the manipulator arms, operating room personnel, and/or external views of the patient.

A microphone 210 is positionable to capture words spoken by the on-site user, and a speaker 212 allows the on-site user to hear audio from the platform, such as verbal communications from the remote user.

The processor 202 is configured to receive input from the cameras 206, 208, and the microphone 210, as well as from the endoscope 10b positioned at the operative site (such as in a body cavity of the patient) by wired or wireless connections. In the illustrated embodiment, a video cable 214 (as a non-limiting example, an HDMI or SDI cable) couples to output from the endoscope. The processor is further configured to transmit signals to the visual display 204 and the speaker 212.

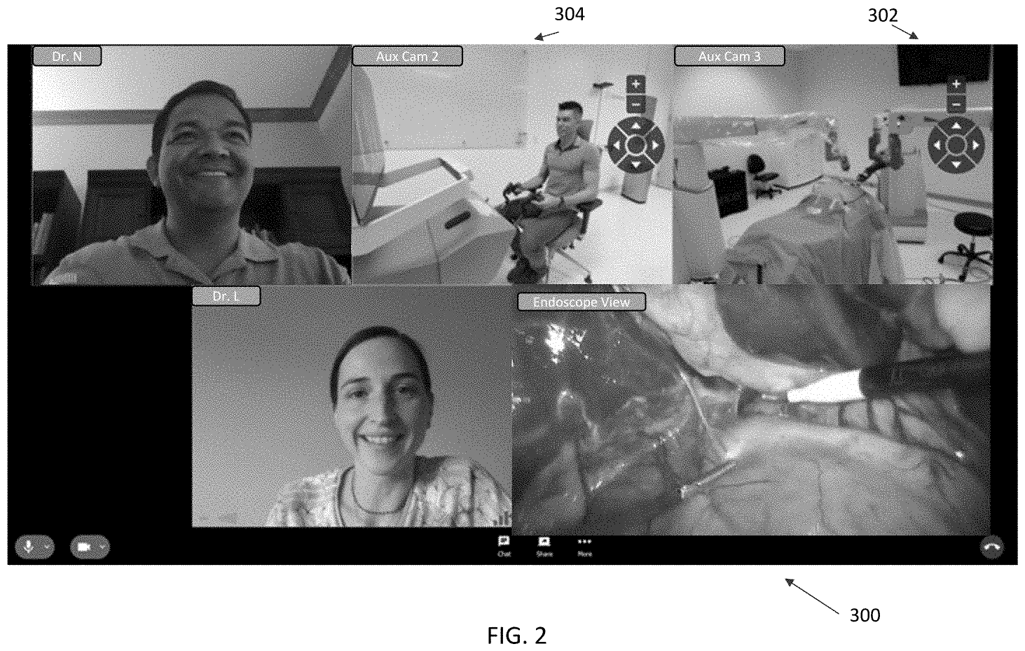

The processor runs a telemedicine videoconference software platform, such as the one marketed by VSee of Sunnyvale, Calif., that enables video conferencing and screen sharing between the on-site surgeon and one or more remote users, each of whom is participating in the videoconference session from a computer, tablet or mobile device having a display and microphone. As shown in FIG. 2, the videoconference display visible to the participants can display one or more of the following: real-time images 300 from the endoscope, images 302 of the operating room (showing, for example, the robotic arms and the patient, as shown) from the camera 206, real-time images 304 from the camera 208 of the on-site surgeon carrying out the surgical procedure, and images of the remote users as captured from cameras connected to their computers, tablets or mobile devices. Images from the other cameras in the operating room, if any, may also be selectively displayed. The remote surgeons can pan, zoom and/or tilt the cameras 206, 208 to change the view of the images captured within the operating room, such as by clicking or tapping the on-screen orientation and zoom icons shown on the display.

The system allows the remote users (and, optionally, the on-site user) to annotate the real-time endoscope images and/or the camera images being shared over the platform using an input device operable with the electronic device they are using to participate in the session. For example, a finger on a touch screen, a stylus, a mouse, keyboard, etc. may be used to annotate the images, allowing the annotations to be seen by the on-site surgeon and other participants as drawings, markings, lines, arrows, text etc. overlays on the endoscopic image. Similarly, a virtual whiteboard may be shared and annotated by the participants.

In some embodiments, the endoscopic images and/or camera images with the annotations may be stored in memory for later use or review. Audio from the session may also be stored in memory, optionally time synced with the endoscopic video feed or the video from one or more of the other cameras. As another example, a recording of the session may be stored in memory, so that all audio, video (including from the endoscope and cameras), whiteboarding and annotations may be viewed simultaneously at a later time.

The system might be further configured to receive and display endoscope images 300 that have been annotated or augmented on the surgeon's display 23 using augmented intelligence features. For example, co-pending application Ser. No. 17/099,761, filed Nov. 16, 2020 and entitled Method and System for Providing Surgical Site Measurement describes a system that analyses images captured by the endoscope and estimates or determines the distances between one or more points in the images (e.g. points identified to the system by the user using an input device). Overlays are generated and displayed on the display to communicate the measured distances to the user. Co-pending U.S. Ser. No. 17/035,534, Method and system for Providing Real Time Surgical Site Measurements describes measuring the extents of, or area of, areas to be treated. The sizing information may be displayed, and in some embodiments, overlays corresponding to size options for surgical mesh that may be used for treatment are displayed to allow the surgeon to evaluate their suitability for the measured site. Co-pending application Ser. No. 16/018,037, filed Dec. 29, 2020 Method of Graphically Tagging and Recalling Identified Structures Under Visualization for Robotic Surgery describes overlays that may be generated over the displayed endoscopic image to identify tagged structures. Co-pending U.S. application Ser. No. 17/368,756, filed Jul. 6, 2021, entitled Automatic Tracking of Target Treatment Sites with Patient Anatomy, describes the use of overlays to mark and keep track of sites (e.g. endometrial sites) for treatment. In other cases, the endoscopic image may be processed to account for illumination deficiencies (such as in, for example, U.S. Ser. No. 17/099,757, filed Nov. 16, 2020), improve image quality, etc. In cases such as those described above, the output from the processor generating the overlays for display with the endoscopic images may be received by the processor 202 so that remote participants will see the same overlays and information that the console display 23 displays for the user.

In the illustrated embodiment, the components of the system 200 are positioned on a cart 216, which may include wheels for easy repositioning within the operating room. In alternative embodiment, some or all of the features of the system may be integrated with a robotic surgical system. For example, any or all of the visual display 204, microphone 210, speaker 212 and camera 208 may integrated with or positioned on the surgeon console of the robotic system. One or more cameras such as camera 206 may be positioned on top of one or more of the manipulator arms of the robotic system, or on another structure in close proximity to the patient bed. In addition, or as an alternative, some components may be mounted to fixtures of the operating room, such as overhead booms or wall mounts.

All prior patents and applications referenced herein, including for purposes of priority, are incorporated herein by reference.

What is claimed is:

1. A method of using a telecollaboration system during surgery, comprising the steps of;
initiating a video conference session between participants, the participants comprising an on-site user in a surgical operating room, and a remote user not in the surgical operating room;
capturing real time images of patient anatomy during a surgical procedure in the operating room and displaying the images in real-time to the video conference participants.

2. The method of claim 1, wherein the real time images are images captured by an endoscope.

3. The method of claim 1, further including capturing second real-time images of at least one of (a) a portion of a manipulator of a robotic surgical system in the operating room; (b) a surgeon in the operating room operating inputs to a robotic surgical system; (c) operating room personnel in the operating room preparing a robotic surgical system, a patient, or surgical devices for surgery,
and displaying the second images in real-time to the video conference participants.

4. The method of claim 3, wherein the second real-time images are displayed simultaneously with the images of patient anatomy.

5. The method of claim 1, further including the step of receiving annotation input from a participant using a user input device, the annotation input comprising annotations to the real time images of the patient anatomy, and displaying the annotations as overlays on the real time images of the patient anatomy.

6. The method of claim 3, further including the step of receiving second annotation input from a participant, the annotation input comprising annotations to the second real time images, and displaying the annotations as overlays on the second real time images.

7. The method of claim 5, further including storing in memory a recording of the real time images showing creation of the annotations.

8. The method of claim 7, wherein the storing step further includes storing audio of verbal communications made during creation of the annotations.

9. The method of claim 2, wherein the real time images are images captured by an endoscope and augmented with overlays.

10. A method of using a telecollaboration system in a surgical operating room, comprising the steps of;
initiating a video conference session between participants, the participants comprising an on-site user in a surgical operating room, and a remote user not in the surgical operating room;
capturing real-time images of at least one of (a) a portion of a manipulator of a robotic surgical system in the operating room; (b) a surgeon in the operating room operating inputs to a robotic surgical system; (c) operating room personnel in the operating room preparing a robotic surgical system, a patient, or surgical devices for surgery, or (d) service personnel performing service on a robotic surgical system, and
displaying the images in real-time to the video conference participants.

11. The method according to claim 10 further including the step of receiving annotation input from a participant, the annotation input comprising annotations to the real time images, and displaying the annotations as overlays on the real time images of the patient anatomy.

12. The method of claim 11, further including storing in memory a recording of the real time images showing creation of the annotations.

13. The method of claim 12, wherein the storing step further includes storing audio of verbal communications made during creation of the annotations.

14. The method of claim 12, further including receiving input given by a remote user using a user input device, and changing a pan, zoom or tilt of one of the cameras in response to the input.
